SEQUENCE LISTING (6 sequences)

SEQ ID NO: 1 - DNA, 558 bp, Homo sapiens
gaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgacgtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaggacgctgcgtctgggaccgctcagcaaggctggcttctacctggccttccaggaccagggtgcctgcatggccctgctatccctgcacctcttctacaaaaagtgcgcccagctgactgtg

SEQ ID NO: 2 - protein, 186 aa, Homo sapiens
EETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTV

SEQ ID NO: 3 - DNA, 1014 bp, Homo sapiens
aacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgccgacccggaggctcctgtgcgccctgcgggggagacctgacttttgaccccggcccccgggacctggtggagccctgggtggtggttcgagggctacgtcctgacttcacctatacctttgaggtcactgcattgaacggggtatcctccttagccacggggcccgtcccatttgagcctgtcaatgtcaccactgaccgagaggtacctcctgcagtgtccgacatccgggtgacgcggtcctcacccagcagcttgagcctggcctgggctgttccccgggcacccagtggggctgtgctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagctggccctg

SEQ ID NO: 4 - protein, 338 aa, Homo sapiens
NLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLAL

SEQ ID NO: 5 - DNA, 1014 bp, Rattus sp.
aacttgacctacttccccgagacggtgcctcgggagctcgtggtgccagtggcaggtagctgcgtggccaatgccgtccctacggccagccccagccccagcctctactgccgggaagatggtcagtgggctgagcagcaggtcacgggctgcagctgcgcgccagggtacgaggccgcggaagggaacaaagtgtgcagagcctgtggccaggggaccttcaagccccaaataggagaagagccctgcctgccatgcccagccaacagccactctaataacattgggtctcctgtctgcctgtgtcggattgggtattaccgggcccgctcggatccccgcagttcgccttgtaccaccccaccctctgctccaagaagcgtagttcaccatctgaatggctcctccctgcgcctggagtggagtgccccccttgagtctggaggccgagaggacctcacttatgctgtacgctgccgagaatgccgtcctgggggttcctgcttgccctgtgggggcgacctgactttcgaccctggtcctcgagacctggttgagccctgggtggcaatccgagggctgcgtcctgatgtcacctatacctttgaggttgctgctttgaatggtgtgtctaccttagccaccggaccacctccttttgagcctgtcaatgtcaccactgaccgtgaagtgcctcccgcggtgtctgacatccgagtcactcggtcctcacccagcagcttgatcctgtcatgggctatccccagagcacccagtggggccgtgctggactacgaggtcaagtatcacgagaagggcgcagagggccctagcagtgtacgtttcctgaagacatcagaaaaccgagctgagctccgggggctgaagcagggagccagctacctggtccaggtccgtgcacggtccgaggccggctatggtccctttggccaagagcatcacagtcagactcaactggatgagagcgagagttggcgggaacagctggccctg

SEQ ID NO: 6 - protein, 338 aa, Rattus sp.
NLTYFPETVPRELVVPVAGSCVANAVPTASPSPSLYCREDGQWAEQQVTGCSCAPGYEAAEGNKVCRACGQGTFKPQIGEEPCLPCPANSHSNNIGSPVCLCRIGYYRARSDPRSSPCTTPPSAPRSVVHHLNGSSLRLEWSAPLESGGREDLTYAVRCRECRPGGSCLPCGGDLTFDPGPRDLVEPWVAIRGLRPDVTYTFEVAALNGVSTLATGPPPFEPVNVTTDREVPPAVSDIRVTRSSPSSLILSWAIPRAPSGAVLDYEVKYHEKGAEGPSSVRFLKTSENRAELRGLKQGASYLVQVRARSEAGYGPFGQEHHSQTQLDESESWREQLAL

(19) United States
(12) Patent Application Publication
(10) Pub. No.: US 2009/0186026 A1
(43) Pub. Date: Jul. 23, 2009

(54) EPHRIN AND EPH RECEPTOR AGONISTS FOR MODULATION OF BONE FORMATION AND RESORPTION

(75) Inventors: Julia Billiard, Collegeville, PA (US); Lioudmila Tchistiakova, Andover, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/354,892

(22) Filed: Jan. 16, 2009

Related U.S. Application Data
(60) Provisional application No. 61/022,002, filed on Jan. 18, 2008.

(51) Int. Cl. A61K 39/395; C07K 16/18

(52) U.S. Cl. 424/134.1; 530/387.3

(57) ABSTRACT

Compositions that stimulate bone formation and inhibit bone resorption through activation of both arms of EphrinB2-EphB4 signaling are provided. The composition comprises a bi-functional molecule having at least one EphB4 ligand-binding domain conjugated to an anti-EphB4 antibody.

### CROSS-REFERENCE TO RELATED APPLICATIONS

This application claims the benefit under 35 U.S.C. §119(e) to co-pending U.S. provisional application No. 61/022,002, filed Jan. 18, 2008, which is hereby incorporated by reference in its entirety.

### FIELD

The invention relates to compositions and methods for increasing bone formation and decreasing bone resorption in the treatment of bone related-disorders. The compositions activate both EphB4 and EphrinB2 activity and/or expression.

### BACKGROUND

Ephrin is a transmembrane ligand that activates signaling of Eph receptor tyrosine kinases. The ephrins, unlike ligands for other receptor tyrosine kinases, must be membrane-anchored to activate Eph receptors. Based on how they are tethered to the membrane, ephrins are divided into two subclasses. The five members of the ephrin-A subclass (ephrin-A1 to -A5) are tethered via a glycosylphosphatidylinositol (GPI) anchor, whereas the three members of the ephrin-B subclass (ephrin-B1 to -B3) have a transmembrane domain and highly conserved cytoplasmic domains. Ephrin-B ligands interact primarily with the B subset of Eph receptors, which consist of at least six members.

EphrinB2 is a transmembrane ligand that is expressed on osteoclasts (OC) whereas EphB4 is a receptor tyrosine kinase expressed on osteoblasts (OB). Zhao et al., Cell Metab. (2006) 4:111-121. Interaction between EphrinB2 and EphB4 results in bi-directional signal transduction: it activates EphrinB2 signaling in OC to inhibit OC differentiation and activates EphB4 pathway in OB to increase OB activity.

Transgenic mice over-expressing EphB4 receptor in OB under control of the Coll promoter exhibit decreased OC number and surface area, enhanced bone formation rate, and increased bone mineral density (BMD). Zhao et al., Cell Metab. (2006) 4:111-121. Interestingly, mice lacking EphrinB2 in the macrophage-osteoclast lineage cells have normal BMD, perhaps due to EphrinB1 or other factors compensating for the absence of EphrinB2.

Available therapies for osteoporosis (e.g., estrogen and related compounds, bisphosphanates, and calcitonin) inhibit bone resorption, but have negligible effects on bone formation. The only agent on the market today that can stimulate new bone formation is parathyroid hormone (PTH), a peptide administered by daily injections. To date, applicants have no knowledge of a therapeutic that can simultaneously stimulate bone formation and inhibit bone resoption.

### SUMMARY

Activation of EphrinB2-EphB4 signaling promotes bone formation and inhibits bone resorption. Compositions of the present invention activate both arms of EphrinB2-EphB4 signaling, and are therefore an anti-resorptive and osteogenic agent capable of treating bone-related disorders.

One aspect of the invention provides a composition comprising at least one EphB4 ligand-binding domain conjugated to an anti-EphB4 antibody, wherein the antibody specifically binds to EphB4.

In another embodiment, the anti-EphB4 antibody comprises an Fc region and is conjugated to the EphB4 ligand-binding domain at the Fc region. In another embodiment, the at least one EphB4 ligand-binding domain conjugated to an anti-EphB4 antibody is expressed as a fusion protein. In another embodiment, the at least one EphB4 ligand-binding domain is conjugated to the anti-EphB4 antibody through a linker.

In another embodiment, the at least one EphB4 ligand-binding domain has a sequence comprising at least about 70% homology to the sequence set forth in SEQ ID NO:2. In another embodiment, the anti-EphB4 antibody binds specifically to an extracellular region of EphB4. In another embodiment, the anti-EphB4 antibody binds specifically to a C-terminal region of the extracellular region of EphB4. In another embodiment, the anti-EphB4 antibody does not bind to an EphB4 ligand-binding domain.

In another embodiment, the anti-EphB4 antibody specifically binds a protein comprising a sequence having at least about 70% homology to the sequence set forth in SEQ ID NO:4 or SEQ ID NO:6. In another embodiment, the anti-EphB4 antibody is a fragment. More particularly, the fragment is a Fab, a Fab′, a F(ab′)₂, an Fv, a dAb or an sdAb. In another embodiment, the anti-EphB4 antibody is monoclonal, polyclonal, chimeric, humanized, recombinant or single chain.

In another embodiment, the composition comprises at least two EphB4 ligand-binding domains conjugated to an anti-EphB4 antibody. In another embodiment, the composition consists essentially of two EphB4 ligand-binding domains conjugated to an anti-EphB4 antibody. Another embodiment further comprises a pharmaceutically acceptable carrier.

Another aspect of the invention provides a composition comprising two EphB4 ligand-binding domains conjugated to an anti-EphB4 antibody specific for a C-terminal portion of an extracellular domain of EphB4, wherein the anti-EphB4 antibody comprises an Fc region and is conjugated to the two EphB4 ligand-binding domains at the Fc region. In another embodiment, the EphB4 ligand-binding domains have a sequence comprising at least about 70% homology to the sequence set forth in SEQ ID NO:2.

Another aspect of the invention provides a method of treating or preventing a bone-related disorder in a subject comprising:

administering to the subject an agent that activates both EphB4 and EphrinB2 signaling or expression, wherein upon administration of the agent, bone formation is increased and bone resorption is decreased.

In another embodiment, the agent comprises an EphB4 ligand-binding domain conjugated to an anti-EphB4 antibody. In another embodiment, the bone-related disorder is selected from the group consisting of osteoporosis, Paget's disease, pycnodysostosis, osteosclerosis, periodontal disease, osteomyelitis, crepitis, arthritis, rickets, bone fracture, bone segmental defects, osteolytic bone disease, osteolytic lesions, primary and secondary hyperparathyroidism, osteomalacia, hyperostosis, osteopetrosis, and osteopenia. In another embodiment, the bone-related disorder is a cancer selected from the group consisting of multiple myeloma, breast cancer and prostate cancer. Another embodiment further comprises administering to the subject a second agent selected from the group consisting of a bisphosphonate, an estrogen, a selective estrogen receptor modulator (SERM), compounds comprising calcium, lanthanide, and calcitonin.

Another aspect of the invention provides a method of increasing bone formation and decreasing bone resorption in a subject, comprising:

administering to the subject an agent that activates both EphB4 and EphrinB2 signaling or expression.

In a more particular embodiment of the method of increasing bone formation and inhibiting bone resorption, the agent comprises an EphB4 ligand-binding domain conjugated to an anti-EphB4 antibody.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 shows an exemplary bivalent molecule for activation of EphrinB2 and EphB4 receptor. Particularly, FIG. 1A shows a schematic representation of the EphB4 receptor where EBD—EphrinB2 binding domain, EC-EBD—extracellular domain without the EphrinB2 binding domain (SEQ ID NOs: 3-6), and TM—transmembrane domain. FIG. 1B shows schematic representation of an exemplary biotherapetic. The molecule consists of two copies of the EphrinB2 binding domain of the human EphB4 receptor (EBD, Sequence ID NO:2) connected by flexible linkers to part of an antibody generated against the C-terminal portion of the extracellular domains of human and rat EphB4 (Sequence ID NO:4 and Sequence ID NO:6). VH —variable region heavy chain; VL —variable region light chain; huFc—constant region heavy chain fragments.

FIG. 2 shows several exemplary variants of the molecule described in FIG. 1. Particularly, FIG. 2A shows EphrinB2-specific antibody used in place of the EphB4 EBD shown in FIG. 1. Inset shows other antibody-derived scaffolds used in any combination to create bi-specific antibodies of this invention. FIG. 2B shows a dual variable domain immunoglobulin containing one EphrinB2 recognition and one EphB4 recognition domain. FIG. 2C shows a single-domain antibody raised in, for example, sharks, camels, or llamas containing EphrinB2 and EphB4 recognition domains. Alternatively, the EphrinB2 and EphB4 recognition domains are positioned on two distinct single-domain antibodies fused by a linker. VH—variable region heavy chain; VL—variable region light chain; CH1—constant region heavy chain 1; CL—constant region light chain; Fab—fragment antigen binding; scFv—single-chain variable fragment; sdAb—single-domain antibody.

FIG. 3 shows expression levels of EphrinB2 and EphB4 in human (A) and mouse (B) osteoblastic cells and in mouse calvaria (B). Production is described in Example 1. Open bars —EphrinB2, hatched bars —EphB4. U2OS osteosarcoma cells do not express detectable levels of EphrinB2.

FIG. 4 demonstrates that a polyclonal anti-EphB4 antibody activates EphB4 receptor in U2OS human osteosarcoma and MC3T3-E1 mouse osteoblast-like cells. The natural EphB4 ligand, EphrinB2, is used as positive control and receptor activation is assessed by measuring autophosphorylation. See Example 2.

FIG. 5 demonstrates that activation of EphB4 signaling leads to mineralized matrix formation in human mesenchymal stem cells (hMSC). The cells were incubated in MSC growth medium containing 0.05 mM ascorbic acid, 10 mM β-glycerophosphate (β-GP) and the indicated amounts of anti-EphB4 antibody. In A, 100 nM dexamethasone (dex) was added to the medium throughout the experiment. After the indicated periods of time, the cells were fixed and subjected to Alizarin red-S staining for mineralized nodule formation.

FIG. 6 shows that a polyclonal anti-EphrinB2 antibody causes EphrinB2 autophosphorylation in (A) SaOS2 human osteosarcoma cells; and (B) MC3T3-E1 mouse osteoblast-like cells.

### DETAILED DESCRIPTION

EphB4 belongs to a family of receptor tyrosine kinases, which is known to be activated by dimerization. Schlessinger J. et al. Cell (2000) 103:211-225. The crystal structure of Ephrin-Eph complexes indicates that the signaling complex consists of a tetramer with two Ephrins and two Eph molecules (Chrencik J E et al. Structure (2006) 14:321-330; and Himanen J P et al., Nature (2001) 414:933-938).

The present invention provides a composition that can both stimulate bone formation and inhibit bone resoption. In particular, the composition comprises a bi-functional molecule that activates Ephrin and EphB receptors, to generate an anti-resorptive and osteogenic effect. The compositions of the present invention are particularly advantageous in their ability to induce EphrinB2-EphB4 signaling. The composition comprises at least one copy of the ligand-binding domain of EphB4 conjugated to an antibody generated against EphB4. More particularly, the molecule consists of two copies of the ligand-binding domain of EphB4 conjugated to an antibody generated against the C-terminal portion of the extracellular domain of EphB4.

### DEFINITIONS

The following abbreviations are used throughout the specification:

| | |
|---|---|
| Ab | antibody |
| BMD | bone mineral density |
| CAT | chloramphenicol transferase |
| CH | constant heavy chain |
| DMEM | Dulbecco's modified Eagle's medium |
| EBD | ephrin binding domain |
| EPH | ephrin |
| GPI | glycophosphatidylinositol |
| HAT | hypoxanthine-aminopterin-thymidine |
| HIC | hydrophobic interaction chromatography |
| OB | osteoblast |
| OC | osteoclast |
| PEG | polyethylene glycol |
| PTH | parathyroid hormone |
| SERM | selective estrogen receptor modulator |
| VH | variable heavy chain |
| VL | variable light chain |

It is to be understood that this invention is not limited to specific compositions or process steps, as such may vary. It should be noted that, as used in this specification and the appended claims, the singular form “a”, “an” and “the” include plural references unless the context clearly dictates otherwise. Thus, for example, reference to “an antibody” includes a plurality of antibodies and reference to “a ligand” includes a plurality of ligands and the like.

The term “antibody” herein is used in the broadest sense and specifically covers monoclonal antibodies (including full length monoclonal antibodies), polyclonal antibodies, multispecific antibodies (e.g., bispecific antibodies), and antibody fragments (described below). It is understood that the term “antibody” as used herein includes within its scope any of the various classes or sub-classes of immunoglobulin derived by conventional and recombinant techniques.

The term “antibody fragments” as used herein refers to fragments of antibodies that retain the principal selective binding characteristics of the whole antibody. Particular fragments are well-known in the art, for example, Fab, Fab′, and F(ab′)₂, which are obtained by digestion with various proteases, pepsin or papain, and which lack the Fc fragment of an intact antibody or the so-called “half-molecule” fragments obtained by reductive cleavage of the disulfide bonds connecting the heavy chain components in the intact antibody. Such fragments also include isolated fragments consisting of the light-chain-variable region, “Fv” fragments consisting of the variable regions of the heavy and light chains, and recombinant single chain polypeptide molecules in which light and heavy variable regions are connected by a peptide linker. Other examples of binding fragments include (i) the Fd fragment, consisting of the VH and CH1, CH2 or CH3 domains; (ii) the dAb fragment (Ward, et al., Nature 341, 544 (1989), the entire disclosures of which are herein incorporated by reference), which consists of a VH domain; (iii) isolated CDR regions; and (iv) single-chain Fv molecules (scFv) described above. In addition, arbitrary fragments that retain antigen-recognition characteristics can be made using recombinant technology.

In addition to antibodies for use in the instant invention, other molecules may also be employed to bind target antigens. Such molecules include small modular immunopharmaceutical (SMIP™) drugs (Trubion Pharmaceuticals, Seattle, Wash.). SMIPs are single-chain polypeptides composed of a binding domain for a cognate structure such as an antigen, a counter receptor or the like, a hinge-region polypeptide having either one or no cysteine residues, and immunoglobulin CH₂ and CH3 domains. SMIPs and their uses and applications are disclosed in, e.g., U.S. Published Patent Appln. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

The term “antigen” as used herein refers to a molecule that induces, or is capable of inducing, the formation of an antibody or to which an antibody binds selectively. “Antigen” is also sometimes referred to as “immunogen”. The anti-EphB4 antibodies of the present invention selectively bind an EphB4 receptor. The term antigen can be used herein interchangeably with the term “target”.

A “bone-related disorder” as used herein refers to a disease or condition directly or indirectly affecting bone formation or bone resorption. Particular bone-related disorders of the present invention include osteoporosis, Paget's disease, pycnodysostosis, osteosclerosis, periodontal disease, osteomyelitis, crepitis, arthritis, rickets, bone fracture, bone segmental defects, osteolytic bone disease, osteolytic lesions, primary and secondary hyperparathyroidism, osteomalacia, hyperostosis, osteopenia and osteopetrosis.

The term “complex” as used herein refers to the association of two or more molecules, by non-covalent bonding; e.g., the association between an antibody and an antigen, or by covalent bonding, either directly or through a linker molecule.

The term “conjugated” as used herein refers to the covalent attachment of two molecules either directly or through a linker. The conjugated molecules can be formed in one step such as through the expression of a single fusion protein, wherein the linker is a series of peptide bonds. Alternatively, conjugated protein molecules can be formed post-translationally through chemical means, such as by cross-linking or through a reactive divalent linker molecule. Examples of suitable linkers for covalent attachment are provided herein.

The “C-terminal region of EphB4 extracellular domain” refers to amino acids 204-540 of human EphB4 (SEQ ID NO:4) or rat EphB4 (SEQ ID NO:6) and constitutes the portion of EphB4 that begins after the EphrinB2 binding domain and ends at the transmembrane domain.

A “derivative” or “analog” may be a polypeptide in which one or more of the amino acid residues are substituted with a conserved or non-conserved amino acid residue (preferably a conserved amino acid residue) and such substituted amino acid residue may or may not be one encoded by the genetic code of a given species, or one in which one or more of the amino acid residues includes a substituent group, or one in which the mature polypeptide is fused with another compound, such as a compound to increase the half-life of the polypeptide (for example, polyethylene glycol), or one in which the additional amino acids are fused to the mature polypeptide, such as a leader or secretory sequence or a sequence which is employed for purification of the mature polypeptide or a protein sequence. Such derivatives and analogs are deemed to be within the scope of those skilled in the art from the teachings herein.

The polypeptides and antibodies of the present invention are preferably provided in an isolated form, and preferably are substantially pure.

Reference to “EphB4” (also designated Htk, HpTK5, Myk1, Mdk2 and Tyro 11) refers to EphB4 receptor tyrosine kinase and naturally occurring variants thereof. EphB4, as it occurs naturally, is a transmembrane protein, with an intracellular and extracellular region. Reference to the “extracellular region of EphB4” and grammatical equivalents, indicates the region of the molecule that would reside outside the cell in the naturally occurring EphB4, irrespective of whether or not the specific composition is in fact naturally occurring in the cell or in an isolated form, such as in a therapeutic.

Reference to “EphB4 ligand-binding domain” refers to at least the section of EphB4 capable of binding the ligand thereof, particularly, the section of EphB4 capable of binding EphrinB2.

“Homology” as used herein indicates the percentage of residues in the amino acid sequence variant that are identical to the residues in a reference sequence after aligning the sequences and introducing gaps, if necessary, to achieve the maximum identical matches between the variant and reference sequences. Methods and computer programs for the alignment are well known in the art. The term “amino acid sequence variant” refers to polypeptides having amino acid sequences that differ to some extent from a native sequence polypeptide. Ordinarily, amino acid sequence variants will possess at least about 70% homology, and preferably, they will be at least about 80%, more preferably at least about 90%, about 95%, about 98% or about 99% homologous with the reference or native protein. The amino acid sequence variants can possess substitutions, deletions, and/or insertions at certain positions within the amino acid sequence of the native or reference amino acid sequence.

“Humanized” forms of non-human (e.g., murine) antibodies are chimeric antibodies which contain minimal sequence derived from non-human immunoglobulin. For the most part, humanized antibodies are human immunoglobulins (recipient antibody) in which hypervariable region residues of the recipient antibody are replaced by hypervariable region residues from a non-human species immunoglobulin (donor antibody), such as mouse, rat, rabbit or non-human primate immunoglobulin having the desired specificity, affinity, and capacity. In some instances, Framework Region (FR) residues of the human immunoglobulin are replaced by corresponding non-human residues to form a humanized antibody. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992), the contents of which are hereby incorporated by reference in their entirety.

The term “inhibition” or grammatical equivalents thereof indicates reduction or elimination of a particular function.

The term “isolated” means that the material is removed from its original environment (e.g., the natural environment if the material is naturally occurring). For example, a naturally-occurring polypeptide present in a living animal is not isolated, but the same polypeptide, separated from some or all of the coexisting materials in the natural system, is isolated. Such polypeptides could be part of a composition, and still be isolated in that some or all of the components such composition are not part of the polypeptide's natural environment.

An “isolated nucleic acid” is a non-naturally occurring nucleic acid sequence, or is a nucleic acid which has the sequence of part or all of a naturally-occurring gene but is free of the sequences that flank the naturally-occurring gene in the genome of the organism in which the gene naturally occurs. The term therefore includes recombinant DNA incorporated into a vector, into an autonomously replicating plasmid or virus, or into the genomic DNA of a prokaryote or eukaryote. The term also includes a separate molecule, such as a cDNA, a genomic fragment, a fragment produced by polymerase chain reaction (PCR) or ligase chain reaction, or a restriction fragment. The term also includes a recombinant nucleotide sequence that is part of a hybrid or chimeric gene; i.e., a gene encoding a fusion protein.

The term “substantially pure” used herein in reference to a given polypeptide means that the polypeptide is substantially free from other biological compounds, such as those in cellular material, viral material, or culture medium, with which the polypeptide may have been associated (e.g., in the course of production by recombinant DNA techniques or before purification from a natural biological source). The substantially pure polypeptide is at least about 75% (e.g., at least about 80, about 85, about 95, or about 99%) pure by dry weight. Purity can be measured by any appropriate standard method, for example, by column chromatography, polyacrylamide gel electrophoresis, or HPLC analysis.

The term “kit” as used herein refers to a packaged set of related components, typically one or more compounds or compositions, and may contain instructions for their use.

The term “linker”, as used herein, refers to a single covalent bond or a series of stable covalent bonds incorporating atoms, such as C, N, O, S and P that covalently attach one molecule to another. Exemplary linkers include a moiety that includes an amino acid, an alkyl group, a substituted alkyl group, a peptide, —C(O)NH—, —C(O)O—, —NH—, —S—, —O—, and the like. Additional linkers suitable for use in the present invention comprise a divalent group of the formula: -L¹-L²-L³-, wherein L¹ is oxo, S(O)_{q}, amino, substituted amino, carbonyl, alkyl, substituted alkyl, alkenyl, substituted alkenyl, alkynyl, substituted alkynyl, cycloalkenyl, substituted cycloalkenyl, heterocyclyl, substituted heterocyclyl, cycloalkyl, substituted cycloalkyl, aryl, substituted aryl, heteroaryl, substituted heteroaryl; L² is absent or oxo, S(O)_{q}, amino, substituted amino, carbonyl, alkyl, substituted alkyl, alkenyl, substituted alkenyl, alkynyl, substituted alkynyl, cycloalkenyl, substituted cycloalkenyl, heterocyclyl, substituted heterocyclyl, cycloalkyl, substituted cycloalkyl, aryl, substituted aryl, heteroaryl, substituted heteroaryl; L³ is absent or oxo, S(O)_{q}, amino, substituted amino, carbonyl, alkyl, substituted alkyl, alkenyl, substituted alkenyl, alkynyl, substituted alkynyl, cycloalkenyl, substituted cycloalkenyl, heterocyclyl, substituted heterocyclyl, cycloalkyl, substituted cycloalkyl, aryl, substituted aryl, heteroaryl, substituted heteroaryl; and q is 0, 1 or 2; and provided that L² is not oxo if either L¹ or L³ is oxo or amino.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Amino” refers to the group —NH₂ or —NH—.

“Substituted amino” refers to the group —NR′R″ where R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R′ and R″ are optionally joined, together with the nitrogen bound thereto to form a heterocyclic or substituted heterocyclic group, provided that R′ and R″ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R′ is hydrogen and R″ is alkyl, the substituted amino group is sometimes referred to herein as alkylamino. When R′ and R″ are alkyl, the substituted amino group is sometimes referred to herein as dialkylamino. When referring to a monosubstituted amino, it is meant that either R′ or R″ is hydrogen but not both. When referring to a disubstituted amino, it is meant that neither R′ nor R″ are hydrogen.

“Aryl” or “Ar” refers to a monovalent aromatic carbocyclic group of from 6 to 14 carbon atoms having a single ring (e.g., phenyl) or multiple condensed rings (e.g., naphthyl or anthryl), which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Alkenyl” refers to alkenyl groups having from 2 to 6 carbon atoms and preferably 2 to 4 carbon atoms and having at least 1 and preferably from 1 to 2 sites of alkenyl unsaturation. Such groups are exemplified, for example, by vinyl, allyl, and but-3-en-1-yl.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy substitution is not attached to a vinyl (unsaturated) carbon atom.

“Alkynyl” refers to alkynyl groups having from 2 to 6 carbon atoms and preferably 2 to 3 carbon atoms and having at least 1 and preferably from 1 to 2 sites of alkynyl unsaturation.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy substitution is not attached to an acetylenic carbon atom.

“Carbonyl” refers to the divalent group —C(O)— which is equivalent to —C(═O)—.

“Cycloalkyl” refers to cyclic alkyl groups of from 3 to 10 carbon atoms having single or multiple cyclic rings including fused, bridged, and spiroring systems. Examples of suitable cycloalkyl groups include, for instance, adamantyl, cyclopropyl, cyclobutyl, cyclopentyl, and cyclooctyl.

“Cycloalkenyl” refers to non-aromatic cyclic alkyl groups of from 3 to 10 carbon atoms having single or multiple cyclic rings and having at least one >C═C<ring unsaturation and preferably from 1 to 2 sites of >C═C<ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heterocycle” or “heterocyclic” or “heterocycloalkyl” or “heterocyclyl” refers to a saturated or unsaturated group having a single ring or multiple condensed rings, including fused bridged and spiroring systems, from 1 to 10 carbon atoms and from 1 to 4 hetero atoms selected from the group consisting of nitrogen, sulfur or oxygen within the ring wherein, in fused ring systems, one or more the rings can be cycloalkyl, aryl or heteroaryl provided that the point of attachment is through the non-aromatic ring.

In one embodiment, the nitrogen and/or sulfur atom(s) of the heterocyclic group are optionally oxidized to provide for the N-oxide, sulfinyl, sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

The term “monoclonal antibody” as used herein refers to an antibody obtained from a population of substantially homogeneous antibodies; i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different antigenic determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies suitable for use in accordance with the present invention may be made by the hybridoma method first described by Kohler et al., Nature 256:495 (1975) the entire disclosure of which is herein incorporated by reference, or may be made by recombinant DNA methods (see, e.g., U.S. Pat. No. 4,816,567, the entire disclosure of which is herein incorporated by reference). “Monoclonal antibodies” may also be isolated from phage antibody libraries using the techniques described in, for example, Clackson et al., Nature 352:624-628 (1991) and Marks et al., J. Mol. Biol. 222:581-597 (1991) the entire disclosure of which is herein incorporated by reference. Monoclonal antibodies specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies [see U.S. Pat. No. 4,816,567; and Morrison et al., Proc. Natl. Acad. Sci. USA 81:6851-6855 (1984)] the entire disclosures of which are herein incorporated by reference.

The terms “protein” and “polypeptide” are used herein in a generic sense to include polymers of amino acid residues, of any length. The term “peptide” is used herein generally to refer to polypeptides having less than 100 amino acid residues. The terms also apply to amino acid polymers in which one or more amino acid residues are an artificial chemical analogue of a corresponding naturally-occurring amino acid, as well as to naturally-occurring amino acid polymers.

The term “specific binding” refers to an affinity between two molecules. For example, specific binding between a molecule and its binding partner results in preferential binding in a heterogeneous sample comprising the molecule and its binding partner and one or more different molecules. Binding in IgG antibodies, e.g., is generally characterized by an affinity of at least about 10⁻⁷ M or higher, such as at least about 10⁻⁸ M or higher, at least about 10⁻⁹ M or higher, at least about 10⁻¹⁰ or higher, at least about 10⁻¹¹ M or higher, or at least about 10⁻¹² M or higher. IgM molecules are known in some instances to have affinities as low as 10⁻⁴.

A “subject” or “patient”, as used herein, refers to a human, a canine, a feline, an ovine, a primate, an equine, a porcine, a caprine, a camelid, an avian, a bovine, an amphibian, a fish, or a murine organism. Most preferably, the subject is a human.

The EphB4 polypeptide sequences (SEQ ID NO: 1 and SEQ ID NO:2), their fragments or other derivatives, or analogs thereof, or cells expressing them can be used as an immunogens to produce antibodies described herein. These antibodies are, for example, polyclonal or monoclonal antibodies. The present invention also includes chimeric, single chain, and humanized antibodies, as well as Fab fragments, or the product of a Fab expression library. Various procedures known in the art may be used for the production of such antibodies and fragments.

In a preferred embodiment, the anti-EphB4 antibodies are specific for particular regions on the EphB4 target antigen and as such, do not bind to other specific regions on the EphB4 antigen. In particular embodiments, the anti-EphB4 antibodies described herein do not bind to the EphB4 ligand-binding domain of EphB4. More particularly still, the anti-EphB4 antibodies bind to the extracellular region of EphB4, specifically, the C-terminal region of EphB4 extracellular domain. Immunogens for production of the aforementioned antibodies include SEQ ID NO:4 and SEQ ID NO:6 and proteins having at least about 70%, about 80%, about 90%, or about 95% homology thereto. Additionally, immunogens for production of the aforementioned antibodies include proteins encoded by SEQ ID NO:3 and SEQ ID NO:5 and nucleotides having at least about 70%, about 80%, about 90%, or about 95% homology thereto Accordingly, in one embodiment, the anti-EphB4 antibodies have specificity for: proteins comprising at least about 70%, about 80%, about 90%, or about 95% homology to SEQ ID NO:4 or SEQ ID NO:6; or proteins encoded by sequences comprising at least about 70%, about 80%, about 90%, or about 95% homology to SEQ ID NO:3 or SEQ ID NO:5.

Antibodies generated against the EphB4 can be obtained by direct injection of the EphB4 receptor (or a portion thereof) into an animal or by otherwise administering the EphB4 receptor to an animal, preferably a nonhuman. The antibody so obtained will then bind the EphB4 receptor itself, preferably with specificity to a particular region, such as the extracellular region. In this manner, even a sequence encoding only a fragment of EphB4 (e.g. the C-terminal region of EphB4 extracellular domain) can be used to generate antibodies binding the whole native protein. Furthermore, the specificity for the particular region, permits only binding to the desired region and permits concomitant activity of the full-length protein (e.g. receptor-ligand binding).

For preparation of monoclonal or polyclonal antibodies, any technique which provides antibodies produced by continuous or multiple cell line cultures can be used. Examples include the hybridoma technique (Kohler and Milstein, 1975, Nature, 256:495-497), the trioma technique, the human B-cell hybridoma technique (Kozbor et al., 1983, Immunology Today 4:72), and the EBV-hybridoma technique to produce human monoclonal antibodies (Cole, et al., 1985, in Monoclonal Antibodies and Cancer Therapy, Alan R. Liss, Inc., pp. 77-96), the entire disclosures of which are herein incorporated by reference.

For example, monoclonal antibodies may be generated by immunizing an animal (e.g., mouse, rabbit, etc.) with a desired antigen and immortalizing the spleen cells from the immunized animal, commonly by fusion with a myeloma cell. Immunization with antigen may be accomplished in the presence or absence of an adjuvant; e.g., Freund's adjuvant. Typically, for a mouse, 10 μg antigen in 50-200 μl adjuvant or aqueous solution is administered per mouse by subcutaneous, intraperitoneal or intra-muscular routes. Booster immunization may be given at intervals; e.g., 2-8 weeks. The final boost is given approximately 2-4 days prior to fusion and is generally given in aqueous form rather than in adjuvant.

Spleen cells from the immunized animals may be prepared by teasing the spleen through a sterile sieve into culture medium at room temperature, or by gently releasing the spleen cells into medium by pressure between the frosted ends of two sterile glass microscope slides. The cells are harvested by centrifugation (400×g for 5 min.), washed and counted. Spleen cells are fused with myeloma cells to generate hybridoma cell lines. Several mouse myeloma cell lines which have been selected for sensitivity to hypoxanthine-aminopterin-thymidine (HAT) are commercially available and may be grown in, for example, Dulbecco's modified Eagle's medium (DMEM) (Gibco BRL) containing 10-15% fetal calf serum. Fusion of myeloma cells and spleen cells may be accomplished using polyethylene glycol (PEG) or by electrofusion using protocols which are routine in the art. Fused cells are distributed into 96-well plates followed by selection of fused cells by culture for 1-2 weeks in 0.1 ml DMEM containing 10-15% fetal calf serum and HAT. The supernatants are screened for antibody production using methods well known in the art. Hybridoma clones from wells containing cells which produce antibody are obtained; e.g., by limiting dilution. Cloned hybridoma cells (4−5×10⁶ cells) are implanted intraperitoneally in recipient mice, preferably of a BALB/c genetic background. Sera and ascites fluids are collected from mice after 10-14 days.

Polyclonal antibodies are produced by immunizing a mouse, rabbit, chicken, or other animal. The polypeptide antigen is injected into the animal along with a suitable adjuvant, such as Freund's adjuvant. Immunization results in the production of antibodies specific to that antigen. The animal serum may be used as the product or the antibodies may be purified from the serum.

The invention also contemplates humanized antibodies which may be generated using methods known in the art, such as those described in U.S. Pat. Nos. 5,545,806; 5,569,825 and 5,625,126, the entire disclosures of which are herein incorporated by reference. Such methods include, for example, generation of transgenic non-human animals which contain human immunoglobulin chain genes and which are capable of expressing these genes to produce a repertoire of antibodies of various isotypes encoded by the human immunoglobulin genes.

Techniques described for the production of single chain antibodies (see, e.g., U.S. Pat. No. 4,964,778, the entire disclosure of which is herein incorporated by reference) can be adapted to produce single chain antibodies to immunogenic polypeptide products of this invention. Also, transgenic mice may be used to express humanized antibodies to immunogenic polypeptide products of this invention.

Polynucleotides may be employed for producing polypeptides or proteins (e.g. EphB4 ligand-binding domain) by recombinant techniques. Thus, for example, the polynucleotide may be included in any one of a variety of expression vectors for expressing a polypeptide. Such vectors include chromosomal, nonchromosomal and synthetic DNA sequences: e.g., derivatives of SV40; bacterial plasmids; phage DNA; baculovirus; yeast plasmids; vectors derived from combinations of plasmids and phage DNA; and viral DNA such as vaccinia, adenovirus, fowl pox virus, and pseudorabies. However, any other suitable vector may be used.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively linked to an appropriate expression control sequence(s) (such as a promoter) to direct mRNA synthesis. As representative examples of such promoters, there may be mentioned: LTR or SV40 promoter; the E. coli. lac or trp; the phage lambda P_{L} promoter and other promoters known to control expression of genes in prokaryotic or eukaryotic cells or their viruses. The expression vector also contains a ribosome binding site for translation initiation and a transcription terminator. The vector may also include appropriate sequences for amplifying expression.

In addition, the expression vectors preferably contain one or more selectable marker genes to provide a phenotypic trait for selection of transformed host cells, such as dihydrofolate reductase or neomycin resistance for eukaryotic cell culture, or tetracycline or ampicillin resistance in E. coli.

The vector containing the appropriate DNA sequence as hereinabove described, as well as an appropriate promoter or control sequence, may be employed to transform an appropriate host to permit the host to express the protein.

As representative examples of appropriate hosts, there may be mentioned: bacterial cells, such as E. coli, Streptomyces, Salmonella typhimurium; fungal cells, such as yeast; insect cells such as Drosophila S2 and Spodoptera Sf9; animal cells such as CHO, COS or Bowes melanoma; adenoviruses; plant cells, etc. The selection of an appropriate host is deemed to be within the scope of those skilled in the art from the teachings herein.

More particularly, the present invention also includes recombinant constructs comprising one or more of the sequences as broadly described herein. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter operably linked to the sequence. Large numbers of suitable vectors and promoters are known to those of skill in the art, and are commercially available. The following vectors are provided by way of example. Bacterial: pQE70; pQE60; pQE-9 (Qiagen); pBS; pD10; phagescript; psiX174; pBluescript SK; pBSKS; pNH8A; pNH16a; pNH18A; pNH46A (Stratagene); ptrc99a; pKK223-3; pKK233-3; pDR540; and pRIT5 (Pharmacia). Eukaryotic: pWLNEO; pSV2CAT; pOG44; pXT1; pSG (Stratagene); pSVK3; PBPV; pMSG; and pSVL (Pharmacia). However, any other suitable plasmid or vector may be used.

Suitable promoter regions can be selected from any desired gene using, for example, CAT (chloramphenicol transferase) vectors or other vectors with selectable markers. Two appropriate vectors are pKK232-8 and pCM7. Particular named bacterial promoters include lacI, lacZ, T3, T7, gpt, lambda P_{R}, P_{L} and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

Desired polypeptides or proteins (e.g. EphB4 ligand-binding domain) can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. Finally, high performance liquid chromatography (HPLC) can be employed for final purification steps.

The polypeptides of the present invention may be a naturally purified product, or a product of chemical synthetic procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated or may be non-glycosylated. Polypeptides of the invention may also include an initial methionine amino acid residue.

Preferred methods for preparation of antibody conjugates described herein involve production of fusion (i.e. chimeric) proteins, wherein genes encoding the antibody (e.g. anti-EphB4) and additional proteins (e.g. EphB4 ligand-binding domains) are concomitantly expressed as one full-length protein. The fusion proteins may comprise an intermediate peptide sequence, which links (i.e. is a “linker” between) the active moieties.

Active moieties can be conjugated by post-translational modifications.

For post-translational attachment, the selection of a linkage to attach the active moieties typically depends on the chemically reactive group on the component to be conjugated. Exemplary reactive groups typically present on the biological or non-biological components include amines, thiols, alcohols, phenols, aldehydes, ketones, phosphates, imidazoles, hydrazines, hydroxylamines, disubstituted amines, halides, epoxides, sulfonate esters, purines, pyrimidines, carboxylic acids, or a combination of these groups. A single type of reactive site may be available on the component (typical for polysaccharides), or a variety of sites may occur (e.g. amines, thiols, alcohols, phenols), as is typical for proteins. Although some selectivity can be obtained by careful control of the reaction conditions, selectivity of is best obtained by selection of an appropriate reactive compound.

In a particular embodiment, the antibody and/or receptor moieties can be conjugated to their binding pairs while attached to an affinity column comprising target antigens or ligands, thereby precluding attachment at the receptor or antigen binding site. In another embodiment, introduction of reactive groups (e.g. azide or alkyne substituents) provides a facet for selective attachment of binding pairs (e.g. through copper catalyzed “click chemistry” rendering a triazole linker).

Additional methods for preparing antibody conjugates include those described for preparation of CMC-544 in U.S. patent application Publication No. 2004-0082764A1 and U.S. patent application Publication No. 2004-0192900, which are incorporated herein with respect to their description of antibody preparation and conjugation. Conjugation may be performed using the following conditions: 10 mg/ml antibody, 8.5% (w/w) calicheamicin derivative, 37.5 mM sodium decanoate, 9% (v/v) ethanol, 50 mM HEPBS (N-(2-Hydroxyethyl)piperazine-N′-(4-butanesulfonic acid)), pH 8.5, 32.degree. C., 1 hour. Hydrophobic interaction chromatography (HIC) may be performed using a butyl sepharose FF resin, 0.65 M potassium phosphate loading buffer, 0.49 M potassium phosphate wash buffer, and 4 mM potassium phosphate elution buffer. Buffer exchange may be accomplished by size exclusion chromatography, ultrafiltration/diafiltration, or other suitable means.

Additional linkers of the present invention comprises a covalent bond, a peptide or a divalent group of the formula: -L¹-L²-L³- as defined above.

The compositions and methods of the invention are useful ex vivo, in vitro, or in vivo. The compounds may be used alone or in compositions together with a pharmaceutically acceptable carrier or excipient. Pharmaceutical compositions of the present invention comprise a therapeutically effective amount of a composition described herein formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants. Other suitable pharmaceutically acceptable excipients are described in The Science and Practice of Pharmacy, Mack Publishing Company, Easton, Pa., 19th Edition (1995), the entire disclosure of which is incorporated herein by reference.

The compounds of the present invention may be administered to humans and other animals enterally or parenterally, such as orally, sublingually, by aerosolization or inhalation spray, rectally, intracisternally, intravaginally, intraperitoneally, intramuscularly, intrathecally, intra-articularly, subcutaneously, intravascularly, bucally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. Transdermal administration can also be accomplished via a transdermal delivery device, such as the RF-Microchannel™ device available from TransPharma Medical™, Ltd. (Northern Ind. Zone, Lod, 71291, Israel). The term parenteral as used herein includes subcutaneous injections, intravenous or intra-arterial, intramuscular, intrasternal injection, or infusion techniques.

Methods of formulation are well known in the art and are disclosed, for example, in Remington: The Science and Practice of Pharmacy, Mack Publishing Company, Easton, Pa., 19th Edition (1995), the entire disclosure of which is incorporated herein by reference. Pharmaceutical compositions for use in the present invention can be in the form of sterile, non-pyrogenic liquid solutions or suspensions, coated capsules, suppositories, lyophilized powders, transdermal patches or other forms known in the art.

Injectable preparations, for example, sterile injectable aqueous or oleaginous suspensions may be formulated according to known techniques using suitable dispersing or wetting agents and suspending agents. The sterile injectable preparation may also be a sterile injectable solution, suspension or emulsion in a nontoxic parenterally acceptable diluent or solvent; for example, as a solution in 1,3-propanediol or 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid are suitable for the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature, and therefore melt in the rectum or vaginal cavity and release the active compound.

Solid dosage forms for oral administration include capsules, tablets, dragees, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, acetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally in a delayed manner. Examples of embedding compositions that can be used include polymeric substances and waxes.

The active compositions can also be in micro-encapsulated form with one or more excipients as noted above. The solid dosage forms of tablets, dragees, capsules, pills, and granules can be prepared with coatings and shells such as enteric coatings, release controlling coatings and other coatings well-known in the pharmaceutical formulating art. In such solid dosage forms the active compound may be admixed with at least one inert diluent such as sucrose, lactose or starch. Such dosage forms may also comprise, as is normal practice, additional substances other than inert diluents; e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially in a certain part of the intestinal tract, optionally in a delayed manner. Examples of embedding compositions that can be used include polymeric substances and waxes.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compositions of the invention may also be formulated for delivery as a liquid aerosol or inhalable dry powder. Liquid aerosol formulations may be nebulized predominantly into particle sizes that can be delivered to the terminal and respiratory bronchioles.

Aerosolized formulations of the invention may be delivered using an aerosol forming device, such as a jet, vibrating porous plate or ultrasonic nebulizer, preferably selected to allow the formation of an aerosol particles having with a mass medium average diameter predominantly between 1 to 5 μm. Further, the formulation preferably has balanced osmolarity ionic strength and chloride concentration, and the smallest aerosolizable volume able to deliver effective dose of the compounds of the invention to the site of the infection. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, ultrasonic nebulizers and energized dry powder inhalers, that are able to nebulized the formulation of the invention into aerosol particle size predominantly in the size range from about 1 to about 5 μm. Preferably, at least about 70% but preferably more than about 90% of all generated aerosol particles are within about 1 to about 5 μm range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AERONEB and AERODOSE vibrating porous plate nebulizers (AeroGen, Inc., Sunnyvale, Calif.), SIDESTREAM nebulizers (Medic-Aid Ltd., West Sussex, England), PARI LC and PARI LC STAR jet nebulizers (Pari Respiratory Equipment, Inc., Richmond, Va.), and AEROSONIC (DeVilbiss Medizinische Produkte (Deutschland) GmbH, Heiden, Germany) and ULTRAAIRE (Omron Healthcare, Inc., Vernon Hills, Ill.) ultrasonic nebulizers.

Compositions of the invention may also be formulated for use as topical powders and sprays that can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott (ed.), “Methods in Cell Biology,” Volume XIV, Academic Press, New York, 1976, p. 33 et seq, the entire disclosure of which is herein incorporated by reference.

Effective amounts of the compositions of the invention for use in treating bone-related disorders generally include any amount sufficient to increase bone formation and decreasing bone resorption in a patient in need of such treatment. The amount of active ingredient that may be combined with the carrier materials to produce a single dosage form will vary depending upon the host treated and the particular mode of administration. It will be understood, however, that the specific dose level for any particular patient will depend upon a variety of factors, including the activity of the specific compound employed, the age, body weight, general health, sex, diet, time of administration, route of administration, rate of excretion, drug combination, and the severity of the particular disease undergoing therapy. The effective amount of the present compounds for a given situation can be readily determined by routine experimentation and is within the skill and judgment of the ordinary clinician.

According to the methods of treatment of the present invention, concomitantly bone formation is increased and bone resorption inhibited in a patient such as a human or lower mammal by administering to the patient an effective amount of a composition of the invention, in such amounts and for such time as is necessary to achieve the desired result. By a “effective amount” of a compound of the invention is meant a sufficient amount of the compound to increase bone formation and inhibit bone resorption, at a reasonable benefit/risk ratio applicable to any medical treatment. It will be understood, however, that the total daily usage of the compounds and compositions of the present invention will be decided by the attending physician within the scope of sound medical judgment.

For purposes of the present invention, an effective dose will generally be a total daily dose administered to a host in single or divided doses in amounts, for example, of from 0.001 to 1000 mg/kg body weight daily and more preferred from 1.0 to 30 mg/kg body weight daily. Dosage unit compositions may contain such amounts or submultiples thereof to make up the daily dose. In general, treatment regimens according to the present invention comprise administration to a patient in need of such treatment from about 10 mg to about 2000 mg of the compound of this invention per day in single or multiple doses.

In another aspect of the invention, kits that include one or more compounds of the invention are provided. Representative kits include a composition of the invention (e.g., at least two EphB4 ligand-binding domains conjugated to an anti-EphB4 antibody or fragment thereof that binds an EphB4 receptor tyrosine kinase) and a package insert or other labeling including directions for treating a condition by administering an effective amount of the compound.

In one embodiment, a kit as used herein comprises a container for containing the compounds or pharmaceutical compositions and may also include divided containers such as a divided bottle or a divided foil packet. The container can be in any conventional shape or form as known in the art which is made of a material which is compatible with the compounds or pharmaceutical compositions held therein, for example a paper or cardboard box, a glass or plastic bottle or jar, a resealable bag (for example, to hold a “refill” of tablets for placement into a different container), or a blister pack with individual doses for pressing out of the pack according to a therapeutic schedule. The container employed can depend on the exact dosage form involved; for example, a conventional cardboard box would not generally be used to hold a liquid suspension. It is feasible that more than one container can be used together in a single package to market a single dosage form. For example, tablets may be contained in a bottle which is in turn contained within a box.

An example of such a container provided with a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory or instructive aid, where the written memory or instructive aid is of the type containing information and/or instructions for the physician, pharmacist or other health care provider, or subject; e.g., in the form of numbers next to the tablets or capsules whereby the numbers correspond with the days of the regimen which the tablets or capsules so specified should be ingested, or a card which contains the same type of information.

Another specific embodiment of a kit is a dispenser designed to dispense the daily doses one at a time in the order of their intended use. Preferably, the dispenser is equipped with a memory or instructive aid, so as to further facilitate compliance with the regimen. An example of such a memory or instructive aid is a mechanical counter, which indicates the number of daily doses that has been dispensed. Another example of such a memory or instructive aid is a battery-powered micro-chip memory coupled with a liquid crystal readout, or audible reminder signal which, for example, reads out the date that the last daily dose has been taken and/or reminds one when the next dose is to be taken.

The kits of the present invention may also comprise, in addition to a compound or composition of the present invention, one or more additional pharmaceutically active compounds. Preferably, the additional compound is another bone-related disorder agent, such as a bisphosphonate, an estrogen, a selective estrogen receptor modulator (SERM), a compound comprising calcium, lanthanide, or calcitonin. The additional compounds may be administered in the same dosage form as the composition of the present invention or in different dosage forms. Likewise, the additional compounds can be administered at the same time as the compound of the present invention or at different times.

The present invention will be understood more readily by reference to the following examples, which are provided by way of illustration and are not intended to be limiting of the present invention.

### EXAMPLES

### Example 1

### Expression of EphB4 and EphrinB2 in Human and Mouse Cells

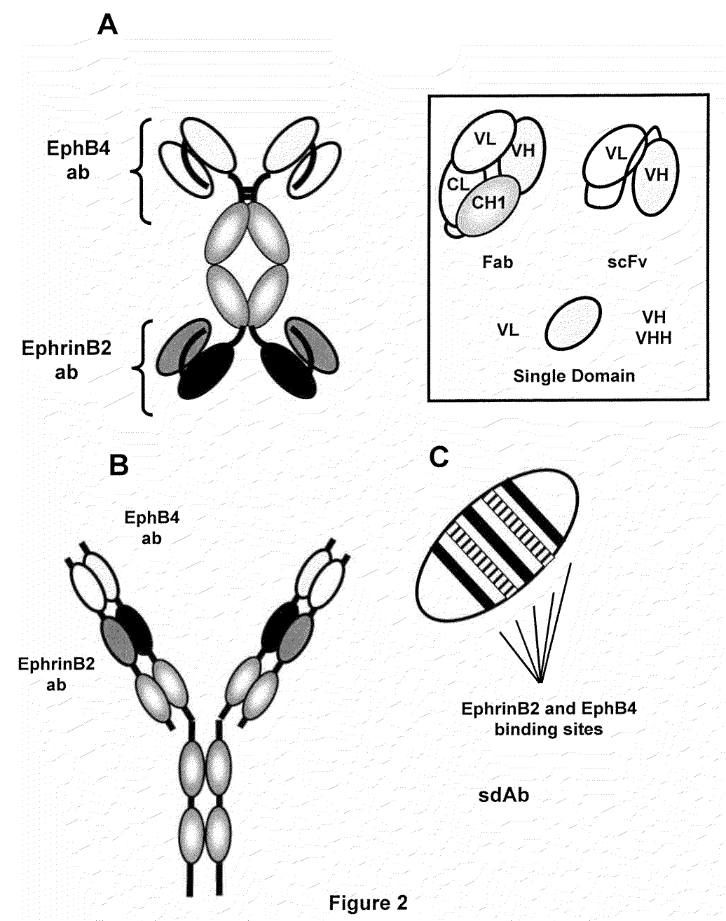

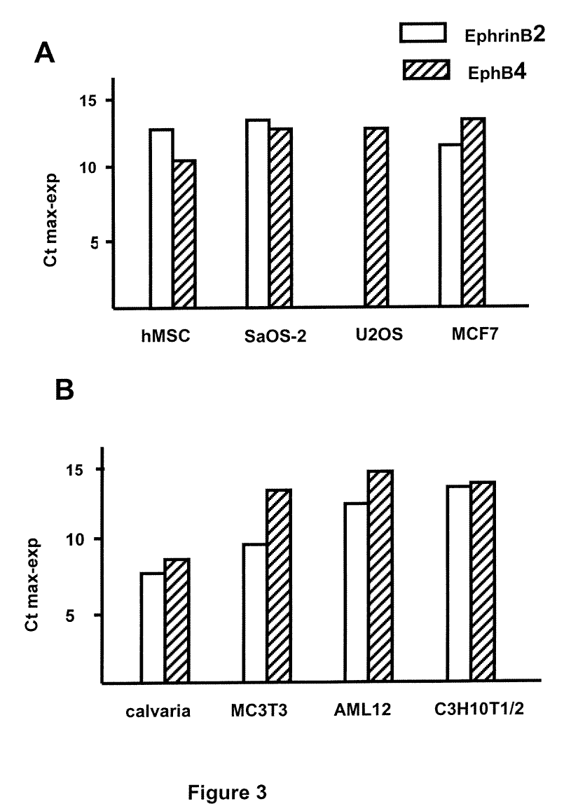

Expression of EphB4 and EphrinB2 in a variety of human (FIG. 3A) and mouse (FIG. 3B) cell lines and tissues was verified. Cells were cultured for 24-48 hours in growth medium and total cellular RNA was isolated using RNeasy Kit (Qiagen, Valencia, Calif.) per manufacturer's instructions. For calvarial RNA, mice were sacrificed and calvarial bones were dissolved in Trizol using a polytron. RNA was extracted with chloroform and purified with RNeasy Kit (Qiagen). All RNA was subjected to real-time RT-PCR analysis using the ABI PRISM 7700 Sequence Detection System and primers and probes purchased from Applied Biosystems Inc. (Foster City, Calif.). The Ct values obtained in each sample were subtracted from the total number of cycles (40) to ensure that higher values reflect higher expression level. As shown in FIG. 2A, EphB4 is expressed in multi-potent human mesenchymal stem cells (hMSC), SaOS2 and U2OS osteosarcoma cells, and MCF7 breast cancer cells. EphrinB2 was expressed in all these cells, except U2OS cells where it is completely undetectable (Ct=40). Both EphB4 and EphrinB2 were expressed in all mouse cell lines tested as well as in mouse calvarial bones (FIG. 3B).

### Example 2

### Anti-EphB4 Antibody Enhances Human Osteoblast Differentiation and Activates Bone Formation

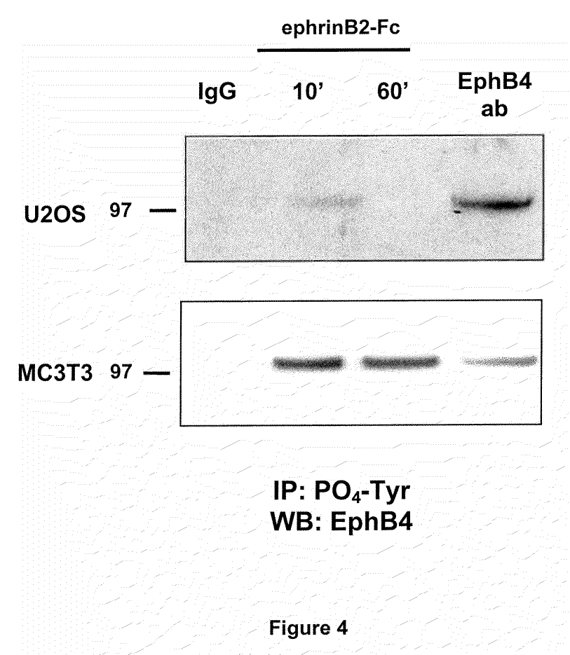

To test if anti-EphB4 antibody activates EphB4 receptor in osteoblastic cells, the ability of this antibody to induce EphB4 autophosphorylation was investigated. As shown in FIG. 4, EphB4 autophoshporylation was induced by its endogenous ligand, EphrinB2. In these experiments, a chimeric protein consisting of the extracellular domain of mouse EphrinB2 fused to the Fc domain of human IgG (EphrinB2-Fc, R&D Systems, Minneapolis, Minn.) was used. EphrinB2-Fc was pre-clustered by incubation with anti-human IgG for 1 h at RT. U2OS human osteosarcoma cells were plated at 60,000/cm² in McCoy's 5A medium containing 10% Cosmic Calf serum (Hyclone, Logan Utah) and 1% penicillin-streptomycin. MC3T3-E1 mouse osteoblast-like cells were plated at 25,000/cm² in MEM containing 10% fetal bovine serum (FBS) and 1% penicillin-streptomycin. Both cell lines were allowed to attach overnight and then switched to their media with 1% serum and incubated for 16-18 h followed by 1 h incubation in serum-free media. Following that incubation, the cells were treated for 10 or 60 min at 37° C. with pre-clustered EphrinB2-Fc, and total cell extracts were precipitated on phospho-tyrosine agarose and probed for EphB4. EphrinB2-Fc induced tyrosine phosphorylation of the endogenous EphB4 receptor, presumably due to receptor autophosphorylation. The much more robust phosphorylation observed in MC3T3 cells was most likely due to the better ability of the mouse EphrinB2 to activate mouse EphB4 receptor. Goat polyclonal antibody raised against the entire extracellular domain of human EphB4 receptor (R&D Systems) also induced receptor autophosphorylation after 1 h incubation (FIG. 4) demonstrating that this antibody can be used to activate EphB4 signaling.

### Effects of Anti-EphB4 Antibody on Osteogenesis:

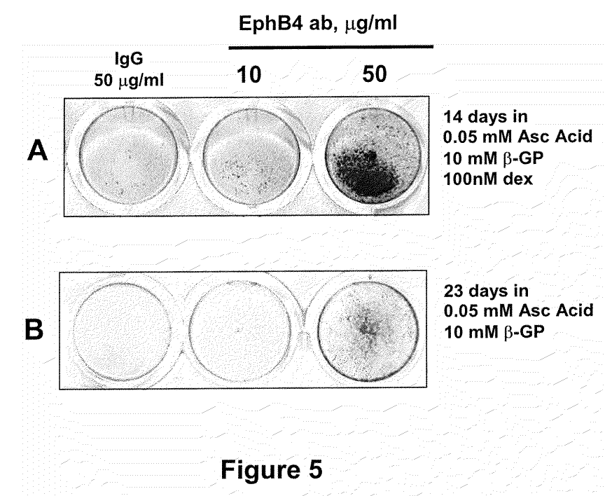

Anti-EphB4 antibody on osteoblastic differentiation of human mesenchymal stem cells (hMSC) was tested. Osteogenesis in this system was assessed by histochemical staining for mineralized matrix formation. Human MSC (Cambrex, Inc., Baltimore, Md.) were plated at 6,000/cm² and allowed to adhere and proliferate overnight in hMSC growth medium (MSCGM; Cambrex). After 24 h, cells were placed in MSCGM supplemented with 0.05 mM ascorbic acid, 10 mM β-glycerophosphate (β-GP), and 100 nM dexamethasone (dex) where indicated. The medium also contained increasing amounts of the goat polyclonal anti-EphB4 antibody (R&D Systems) or non-specific IgG. When the known osteogenic agent, dex, was present in culture, the EphB4 activating antibody strongly enhanced dex-induced osteoblastic differentiation and resulted in faster and greater extent of matrix mineralization (FIG. 5A). After 23 days of incubation, anti-EphB4 antibody was analyzed and resulted in mineralized matrix formation in absence of dex (FIG. 5B), indicating that EphB4 activation induces osteogenic commitment and differentiation of pluripotent human mesenchymal stem cells.

### Example 3

### The Extracellular Domain of EphB4 Receptor Inhibits Human Osteoclast Differentiation

Recombinant mouse EphB4-Fc chimera or human Fc were pre-incubated with anti-human IgG for 1 h at RT. Cells were incubated in 1% serum overnight followed by serum-free medium for 45 min prior to addition of treatments. Cells were then treated at 37 C with the pre-conjugated EphB4-Fc or Fc (4 μg/ml each) for the indicated times. SaOS2 cells were also treated with 50 μg/ml of anti-EphrinB2 antibody for 1 h. At the end of treatment, total cell lysates were isolated and subjected to immunoblotting with the indicated antibodies (SaOS2 cells: 45 μg/lane for and 15 μg/lane for total EphrinB2; MC3T3-E1 cells: 24 μg/lane for PO₄-Ephrin and 7 μg/lane for EphrinB2).

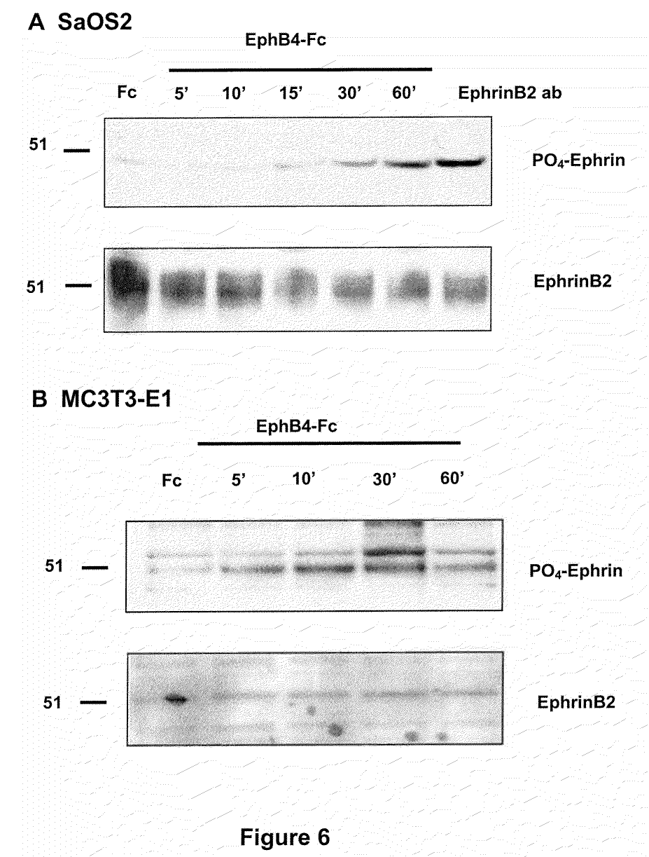

The ability of commercially available recombinant extracellular domain of mouse EphB4-Fc (EphB4EC-Fc, 91% identity to human within the EphrinB2 binding domain, R&D Systems) to activate EphrinB2 signaling was tested. EphB4EC-Fc was pre-clustered by incubation with anti-human IgG for 1 h at RT. As shown in FIG. 6A, treatment of SaOS2 human osteosarcoma cells with pre-conjugated EphB4EC-Fc led to a time-dependent increase in the tyrosine phosphorylation of EphrinB. Since EphB4EC binds only EphrinB2, the observed band likely reflects EphrinB2 phosphorylation. In contrast, treatment with pre-clustered human Fc had no effect (FIG. 6A). Thus, the extracellular domain of EphB4 has the ability to activate EphrinB2 signaling in human osteoblastic cells. Anti-EphrinB2 antibody was also capable of stimulating EphrinB2 phosphorylation (FIG. 6A). Neither EphB4EC nor EphrinB2 antibody had any effect on the total levels of EphrinB2 in the cells (FIG. 6A, bottom panel). Similar to its effects on human EphrinB2, EphB4EC-Fc also promoted tyrosine phosphorylation of EphrinB2 in mouse MC3T3-E1 osteoblast-like cells (FIG. 6B). The time course differed somewhat from that in human cells with the maximal phosphorylation observed at 30 min.

The ability of the EphB4EC-Fc to inhibit osteoclastic differentiation of human CD14+ monocytes in presence of MCSF and RANKL is assessed. Since this protein has been shown to inhibit osteoclast differentiation in mouse systems (Zhao et al., Cell Metab. (2006) 4:111-121), similar effects are expected in human osteoclasts.

### Example 4

### Simultaneous Activation of EphrinB2 and EphB4 Receptor Leads to Enhanced Bone Formation

Effects of bivalent molecules on bone formation and resorption are monitored in a mouse calvarial organ culture assay. In this assay, the rate of bone formation is assessed by directly measuring total bone area as well as by counting the number of bone-forming osteoblasts. Bone resorption rate is assessed by measuring the amount of calcium released into the medium upon treatment with 100 ng/ml RANKL, 5 μg/ml IL-1 alpha, or 100 nM PTH. Real-time RT-PCR demonstrated that both EphrinB2 and EphB4 are expressed in mouse calvarial bones (FIG. 3B). Therefore, polyclonal antibodies raised in goat against the entire extracellular domains of mouse EphrinB2 and EphB4 receptor (R&D Systems) will be used to activate EphrinB2 and EphB4 signaling. Non-specific goat IgG (R&D Systems) will be used as control. Activation of EphrinB2 is expected to result in decreased bone resorption whereas activation of EphB4 should increase bone formation. The two antibodies are then conjugated to each other to create an anti-Ephrin2-EphB4 chimera. The potency of this bivalent chimera is compared to the two individual antibodies in the same experiment. More specifically, a dose response (5-20 μg/ml) for anti-EphB4 or anti-EphrinB2 antibodies, for an equimolar amount of the anti-Ephrin2-EphB4 chimera, or for non-specific IgG for control is performed.

### Example 5

### Generating a Bifunctional Molecule to Activate EphrinB2 and EphB4 Receptor

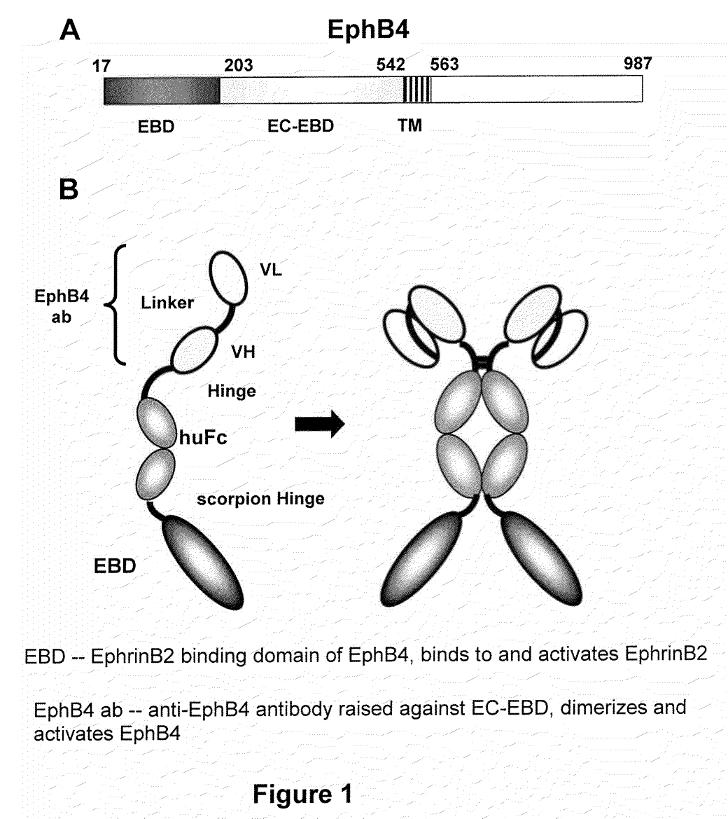

The domain structure of EphB4 receptor is shown in FIG. 1A. An exemplary bivalent molecule for simultaneous activation of EphrinB2 and EphB4 receptor is shown in FIG. 1B. The molecule consists of two copies of the EphrinB2 binding domain of the human EphB4 receptor (EBD, Sequence ID NO:2) connected by flexible linkers to part of an antibody generated against the C-terminal portion of the extracellular domains of human and rat EphB4 (EC-EBD, Sequence ID NO:4 and Sequence ID NO:6). The domain lacking EBD is used for antibody generation to avoid the antibody portion of the construct binding the EBD within the same molecule. Since the EphB4 receptor recognizes EphrinB2 only (Chrencik et al., J Biol Chem (2006) 281:28185-28192), the construct is expected to have very high specificity. Instead of a portion of the anti-EphB4 antibody, the full-length antibody can also be used. Furthermore, the EBD can be replaced by a full-length anti-EphrinB2 antibody or a portion thereof as shown in FIG. 2A. FIGS. 2B and 2C show other exemplary approaches to generating a bifunctional molecule to activate EphrinB2 and EphB4 receptor, including a dual-variable domain immunoglobulin and a single-domain antibody.

### Example 6

### Characterization of the Bi-Functional Molecules

The bi-functional molecules described in Example 4 are characterized in vitro, ex vivo, and ultimately in vivo. For in vitro characterization, osteoblastic cell lines expressing EphrinB2 and/or EphB4 identified in FIG. 3A are used to ensure that the construct enhances osteoblast differentiation and/or function. In parallel, CD14+ cells differentiated to osteoclasts are used to test the ability of the construct to inhibit osteoclastogenesis. The mouse calvarial assay described in Example 4 is used to test the ability of the molecule to act on both bone formation and resorption in organ culture. Finally, ovariectomized rat models are used to test performance of this biotherapeutic in animals.

### SEQUENCES

The following sequences, provided in the enclosed sequence listing, are cited throughout the application:

SEQ ID NO:1. The nucleotide sequence of the EphrinB2 binding domain of human EphB4.

SEQ ID NO:2. The amino acid sequence of the EphrinB2 binding domain of human EphB4.

SEQ ID NO:3. The nucleotide sequence of the human EphB4 extracellular domain without the EphrinB2 binding domain.

SEQ ID NO:4 The amino acid sequence of the human EphB4 extracellular domain without the EphrinB2 binding domain.

SEQ ID NO:5. The nucleotide sequence of the rat EphB4 extracellular domain without the EphrinB2 binding domain.

SEQ ID NO:6. The amino acid sequence of the rat EphB4 extracellular domain without the EphrinB2 binding domain.

It will be apparent to those skilled in the art that various modifications and variation can be made to the compositions and methods described herein and as provided in the examples above without departing from the spirit or scope of the compositions and methods.

All cited patents and publications referred to in this application are herein incorporated by reference in their entirety.

1. A composition comprising at least one EphB4 ligand-binding domain conjugated to an anti-EphB4 antibody, wherein the antibody specifically binds to EphB4.

2. The composition of claim 1, wherein the anti-EphB4 antibody comprises an Fc region and is conjugated to the EphB4 ligand-binding domain at the Fc region.

3. The composition of claim 1, wherein the at least one EphB4 ligand-binding domain conjugated to an anti-EphB4 antibody is expressed as a fusion protein.

4. The composition of claim 1, wherein the at least one EphB4 ligand-binding domain is conjugated to the anti-EphB4 antibody through a linker.

5. The composition of claim 4, wherein the linker comprises a covalent bond, a peptide or a divalent group of the formula: -L¹-L²-L³-, wherein L¹ is oxo, S(O)_{q}, amino, substituted amino, carbonyl, alkyl, substituted alkyl, alkenyl, substituted alkenyl, alkynyl, substituted alkynyl, cycloalkenyl, substituted cycloalkenyl, heterocyclyl, substituted heterocyclyl, cycloalkyl, substituted cycloalkyl, aryl, substituted aryl, heteroaryl, substituted heteroaryl; L² is absent or oxo, S(O)_{q}, amino, substituted amino, carbonyl, alkyl, substituted alkyl, alkenyl, substituted alkenyl, alkynyl, substituted alkynyl, cycloalkenyl, substituted cycloalkenyl, heterocyclyl, substituted heterocyclyl, cycloalkyl, substituted cycloalkyl, aryl, substituted aryl, heteroaryl, substituted heteroaryl; L³ is absent or oxo, S(O)_{q}, amino, substituted amino, carbonyl, alkyl, substituted alkyl, alkenyl, substituted alkenyl, alkynyl, substituted alkynyl, cycloalkenyl, substituted cycloalkenyl, heterocyclyl, substituted heterocyclyl, cycloalkyl, substituted cycloalkyl, aryl, substituted aryl, heteroaryl, substituted heteroaryl; and q is 0, 1 or 2; and provided that L² is not oxo if either L¹ or L³ is OXO or amino.

6. The composition of claim 1, wherein the at least one EphB4 ligand-binding domain has a sequence comprising at least about 70% homology to the sequence set forth in SEQ ID NO:2.

7. The composition of claim 1, wherein the anti-EphB4 antibody binds specifically to an extracellular region of EphB4.

8. The composition of claim 7, wherein the anti-EphB4 antibody binds specifically to a C-terminal region of the extracellular region of EphB4.

9. The composition of claim 1, wherein the anti-EphB4 antibody does not bind to an EphB4 ligand-binding domain.

10. The composition of claim 1, wherein the anti-EphB4 antibody specifically binds: a protein comprising a sequence having at least 70% homology to the sequence set forth in SEQ ID NO:4 or SEQ ID NO:6 or a protein encoded by a nucleotide sequence having at least 90% homology to the sequence set forth in SEQ ID NO:3 or SEQ ID NO:5.

11. The composition of claim 1, wherein the anti-EphB4 antibody is a fragment.

12. The composition of claim 11, wherein the fragment is a Fab, a Fab′, a F(ab′)₂, an Fv, a dAb, or an sdAb.

13. The composition of claim 1, wherein the anti-EphB4 antibody is monoclonal, polyclonal, chimeric, humanized, recombinant or single chain.

14. The composition of claim 1, comprising at least two EphB4 ligand-binding domains conjugated to an anti-EphB4 antibody.

15. The composition of claim 1, consisting essentially of two EphB4 ligand-binding domains conjugated to an anti-EphB4 antibody.

16. The composition of claim 1, further comprising a pharmaceutically acceptable carrier.

17. A composition comprising two EphB4 ligand-binding domains conjugated to an anti-EphB4 antibody specific for a C-terminal portion of an extracellular domain of EphB4, wherein the anti-EphB4 antibody comprises an Fc region and is conjugated to the two EphB4 ligand-binding domains at the Fc region.

18. The composition of claim 17, wherein the EphB4 ligand-binding domains have a sequence comprising at least about 70% homology to the sequence set forth in SEQ ID NO:2.

19. A method of treating or preventing a bone-related disorder in a subject comprising: administering to the subject an agent that activates both EphB4 and EphrinB2 signaling or expression, wherein upon administration of the agent, bone formation is increased and bone resorption is decreased.

20. The method of claim 19, wherein the agent comprises an EphB4 ligand-binding domain conjugated to an anti-EphB4 antibody.

21. The method of claim 19, wherein the bone-related disorder is selected from the group consisting of osteoporosis, Paget's disease, pycnodysostosis, osteosclerosis, periodontal disease, osteomyelitis, crepitis, arthritis, rickets, bone fracture, bone segmental defects, osteolytic bone disease, osteolytic lesions, primary and secondary hyperparathyroidism, osteomalacia, hyperostosis, osteopenia and osteopetrosis.

22. The method of claim 19, wherein the bone-related disorder is a cancer selected from the group consisting of multiple myeloma, breast cancer and prostate cancer.

23. The method of claim 19, further comprising administering to the subject a second agent selected from the group consisting of a bisphosphonate, an estrogen, a selective estrogen receptor modulator (SERM), calcium, lanthanide, and calcitonin.

24. A method of increasing bone formation and decreasing bone resorption in a subject, comprising:
administering to the subject an agent that activates both EphB4 and EphrinB2 signaling or expression.

25. The method of claim 24, wherein the agent comprises an EphB4 ligand-binding domain conjugated to an anti-EphB4 antibody.
